(19) United States
(12) Patent Application Publication
(10) Pub. No.: US 2026/0263347 A1
(43) Pub. Date: Sep. 10, 2026

(54) HIGH PERFORMANCE FRAGRANCE COMPOSITIONS FOR RINSE-OFF CONDITIONER

(71) Applicant: International Flavors & Fragrances Inc., New York, NY (US)

(72) Inventors: Clementine Marteau-Roussy, Lille (FR); Frederique Terranova, Carry le Rouet (FR)

(21) Appl. No.: 19/163,433

(22) PCT Filed: Mar. 14, 2024
(86) PCT No.: PCT/US2024/019887

§ 371 Date: Sep. 9, 2025

Related U.S. Application Data
(60) Provisional application No. 63/490,343, filed on Mar. 15, 2023.

(51) Int. Cl. A61K 8/92; A61K 8/35; A61K 8/37; A61Q 5/12; A61Q 13/00; C11B 9/00

(52) U.S. Cl. CPC A61K 8/922; A61K 8/35; A61K 8/37; A61Q 5/12; A61Q 13/00; C11B 9/00

(57) ABSTRACT

Disclosed is a high performance fragrance composition, which provides long lasting performance on dry hair. Also disclosed are hair rinse-off conditioner products containing such a fragrance composition.

### BACKGROUND

Attempts have been made to design fragrance accords and fragrance compositions suitable for specific product applications that have superior or enhanced properties leading to consumer benefits. For example, WO1999/065458 defines two categories of fragrance ingredients to be formulated into high impact accords based on a combination of boiling point, partition coefficient (ClogP) and measured odor detection thresholds. These accords can be optionally encapsulated in starch to provide a boost of fragrance from a detergent powder when dispersed in water. U.S. Pat. No. 9,222,055 provides a high intensity fragrance which includes 75% to 100% of at least two key fragrance ingredients having boiling points between 100° C. and 300° C. at a pressure of 760 mmHg, molecular weights within the range of 70 atomic mass units to 175 atomic mass units, and ClogP values between 0 and 4. US2005/0003975 also discloses perfume compositions composed of high impact accord perfume ingredients classified by boiling point, calculated CLogP, and odor detection thresholds. EP196454 describes fragrance compositions for use in consumer products to reduce allergic reactions.

In spite of the many attempts described above, there is still a need to design high performing and long lasting fragrances, in particular on dry hair. The present invention addresses this need in the art.

### SUMMARY OF THE INVENTION

This invention is a high performance fragrance composition composed of at least five fragrance ingredients each having an analytical deposition value and retention value, wherein the combined analytical deposition value and retention value of each fragrance falls with a range set forth by formula (I):

$$60 \% \leq R + 5 D \leq 150 \% , \qquad ( I )$$

wherein “R” is the experimentally determined percent retention of the fragrance on a dry hair sample after 10 hours and “D” is an experimentally determined percent deposition of the fragrance on a wet hair sample, wherein the at least five fragrances include:
(i) at least two fragrance ingredients having an intensity value in the range of 40 to 60, wherein each of the fragrance ingredients is present at a dosage greater than 2 wt % of the high performance fragrance composition, and
(ii) at least three fragrance ingredients having an intensity value greater than 60, wherein each of the fragrance ingredients is present at a dosage greater than 1 wt % of the high performance fragrance composition, wherein the intensity value of (i) and (ii) is determined on a scale of 0 to 100.

In one aspect, the high performance fragrance composition includes at least two to four fragrance ingredients having an intensity value in the range of 40 to 60, wherein each of the fragrance ingredients is present at a dosage greater than 2 wt % of the fragrance composition.

In other aspects, the high performance fragrance composition includes at least two to four fragrance ingredients having an intensity value in the range of 40 to 60, wherein each of said fragrance ingredients is present at a dosage greater than 5 wt % of the fragrance composition.

In further aspects, the high performance fragrance composition includes at least three to five fragrance ingredients having an intensity value greater than 60, wherein each of the fragrance ingredients is present at a dosage greater than 1 wt % of the fragrance composition.

In still further aspects, the high performance fragrance composition includes at least two to four fragrance ingredients having an intensity value greater than 60, wherein each of the fragrance ingredients is present at a dosage greater than 4 wt % of the fragrance composition.

A hair rinse-off conditioner product including a product base and a high performance fragrance composition is also provided.

### DETAILED DESCRIPTION OF THE INVENTION

As used herein, the terms “comprises”, “comprising”, “includes”, “including”, “has”, “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are used to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

This invention is a high performance fragrance composition that exhibits long lasting performance on dry hair for at least 10 hours. Fragrances were screened for their deposition and retention on hair swatches. A set of fragrances were shown to exhibit both suitable deposition and retention on dry hair for at least 10 hours and is defined by the analytical deposition and retention values using formula (I):

$$60 \% \leq R + 5 D \leq 150 \% \qquad ( I )$$

wherein “R” is the experimentally determined percent retention of the fragrance on a dry hair sample after 10 hours and “D” is the experimentally determined percent deposition of the fragrance on a wet hair sample. The perceived intensity (scale of 0-100) of fragrances within this class (i.e., fragrances defined by formula (I)) was then evaluated in rinse-off conditioner applications and certain fragrances were shown to provide fragrance formulations with superior intensity on dry hair. Based on this analysis, a set of key characteristics were developed to produce a high performance fragrance, and a rinse-off conditioner product containing the same.

Accordingly, this invention provides a high performance fragrance composition, and rinse-off conditioner product containing the same, the fragrance composition including at least five fragrance ingredients, each fragrance ingredient having an analytical deposition value and retention value, wherein the combined analytical deposition value and retention value of each fragrance falls with a range set forth by formula (I):

$$60 \% \leq R + 5 D \leq 150 \% , \qquad ( I )$$

wherein “R” is the experimentally determined percent retention of the fragrance on a dry hair sample after 10 hours and “D” is the experimentally determined percent deposition of the fragrance on a wet hair sample. In some aspects, the high performance fragrance composition, and rinse-off conditioner product containing the same, include at least five, at least six, at least seven, at least eight, at least nine, or at least 10 or more fragrance ingredients having a combined analytical deposition value and retention value falling with range set forth by formula (I).

In certain aspects, the at least five fragrances included in the high performance fragrance composition are further defined by their intensity values. In particular, the at least five fragrances comprises:
(i) at least two fragrance ingredients having an intensity value in the range of 40 to 60, wherein each of these fragrance ingredients is present at a dosage greater than 2 wt % of the high performance fragrance composition, and
(ii) at least three fragrance ingredients having an intensity value greater than 60, wherein each of these fragrance ingredients is present at a dosage greater than 1 wt % of the high performance fragrance composition.

As used herein, the term “high performance fragrance composition,” “fragrance composition,” “fragrance formulation,” “perfume composition,” “fragrance accord,” or “perfume accord” is a composition composed of at least five fragrances, wherein the fragrance composition is intended for use in a rinse-off conditioner application, i.e., to impart a pleasant odor thereto, or cover a malodor thereof.

As used herein, the terms “perfume,” “fragrance”, “fragrance material” and “fragrance ingredient” are used interchangeably to designate the component in the high performing fragrance composition that is capable of imparting or modifying the odor of hair or other substrates. A “fragrance ingredient” can encompass any suitable perfume raw material for fragrance uses, including materials such as, for example, alcohols, aldehydes, ketones, esters, ethers, acetates, nitriles, terpene hydrocarbons, nitrogenous or sulfurous heterocyclic compounds and essential oils. Non-limiting examples of the fragrance materials can also include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH change, or by enzymatic release. Individual fragrance ingredients are readily available from commercial sources such as International Flavor & Fragrances (New York, NY), Givaudan Fragrances Corp. (East Hanover, NJ), Symrise (Branchburg, NJ), and Firmenich (Princeton, NJ).

“Neat” or “neat oil” refers to a fragrance composition that is free from extraneous matter. A neat fragrance composition includes only fragrance ingredients and is unencapsulated and/or unbound from other compounds that would cause a delay in the release of the fragrance ingredients.

As used herein, a fragrance ingredient selected for use in a high performance fragrance composition is defined as a fragrance ingredient exhibiting both a suitable level of deposition on wet hair and a high level of retention for at least 10 hours on dry hair. In particular aspects, a fragrance ingredient selected for use in a high performance fragrance composition exhibits at least 1% to 25% deposition on wet hair, e.g., at least 1%, 5%, 7%, 10%, 12%, 15%, 17%, 20%, 22% or 25% deposition on wet hair. The level of deposition of a fragrance is determined by uniformly applying a rinse-off conditioner product including the fragrance ingredient to a hair sample and measuring the amount of fragrance deposited on the hair sample after rinsing the hair sample with water to remove residual product. Fragrance measurements are made by extracting fragrance from the wet hair sample with solvent and comparing the amount of extracted fragrance, e.g., determined by gas chromatography, to one or more control samples.

In addition to suitable level of deposition, a fragrance ingredient selected for use in a high performance fragrance composition exhibits at least 60% retention on dry hair for at least 10 hours, e.g., at least 60%, 65%, 70%, 75%, 80%, 85%, 90%, 95% or 100% retention on dry hair for at least 10 hours. The level of retention of a fragrance is determined by uniformly applying a rinse-off conditioner product including the fragrance ingredient to a hair sample, measuring the amount of fragrance deposited on the hair sample after rinsing the hair sample with water to remove residual product, allowing the hair sample to dry, and after 10 hours measuring the amount of fragrance retained on the dry hair sample. Fragrance measurements are made as described above and the fragrance retention is determined with respect to the amount of fragrance deposited, e.g., a loss of 5% fragrance after 10 hours indicates that 95% of the fragrance deposited was retained.

Fragrance ingredients having a combined analytical deposition value and retention value falling with range set forth by formula (I) are provided in Table 1. Notably, the fragrance ingredients listed in Table 1 are not considered bottom notes that perfumers typically select for providing long lasting performance on dry hair.

TABLE 1

| Fragrance Ingredient | CAS NO. | R | D | R + 5D |
|---|---|---|---|---|
| DIMETH BENZ CARB | 10094-34-5 | 17.2 | 35 | 121 |
| BUTY | | | | |
| ROSETONE | 90-17-5 | 6.4 | 33 | 65 |
| BACDANOL | 28219-61-6 | 7 | 60 | 95 |
| CYCLAMAL | 103-95-7 | 9.8 | 39 | 88 |
| BOURGEONAL | 18127-01-0 | 4.9 | 91 | 115.5 |
| LILIAL | 80-54-6 | 5.3 | 96 | 122.5 |
| ALD C-16 | 77-83-8 | 10.5 | 32 | 84.5 |
| STARFLEUR | 56836-93-2 | 12.4 | 58 | 120 |
| ALD C-12 MNA | 110-41-8 | 8.4 | 66 | 108 |
| ARCTICAL | 61668-40-4 | 12.5 | 23 | 85.5 |
| DECALACTONE GAMMA | 706-14-9 | 11.5 | 38.4 | 95.9 |
| THYMOL | 89-83-8 | 12.2 | 57 | 118 |
| OPALENE | 174155-47-6 | 10 | 65 | 115 |
| VANITROPE | 94-86-0 | 19.2 | 28 | 124 |
| SALICYNALVA | 3508-98-3 | 8.6 | 60 | 103 |
| GALBASCONE | 56973-85-4 | 11.7 | 43 | 101.5 |
| BENZ BENZOATE | 120-51-4 | 13.6 | 57 | 125 |
| PATCHOULI OIL | 9/3/8014 | 20 | 13 | 113 |
| CYCLAPROP | 67634-24-6 | 12.3 | 39 | 100.5 |
| KHUSINIL | 75490-39-0 | 7.5 | 31 | 68.5 |
| INDOLE | 120-72-9 | 14 | 24 | 94 |
| UNDECALACTONE | 104-67-6 | 12.6 | 41 | 104 |
| GAMMA | | | | |
| DAMASCONE ALPHA | 24720-09-0 | 4.1 | 52 | 72.5 |
| ALD C-11 INTRELEVEN | 143-14-6 | 17.4 | 26 | 113 |
| RHUBOFIX | 41816-03-9 | 6.1 | 56 | 86.5 |
| PHARAONE | 313973-37-4 | 18.9 | 29 | 123.5 |
| DAMASCONE DELTA | 71048-82-3 | 8.6 | 23.7 | 66.7 |
| AMYL CINN ALD | 78605-96-6 | 12 | 64 | 124 |
| ETH-2,4-DECADIENOATE | 3025-30-7 | 17.8 | 23.4 | 112.4 |
| ISO BUTYL QUINOLINE | 65442-31-1 | 15.8 | 39 | 118 |
| ALD C-12 LAURIC | 112-54-9 | 13 | 30 | 95 |

In addition to meeting the criteria set forth in formula (I), at least two fragrance ingredients of the high performance fragrance composition have an intensity value in the range of 40 to 60, and at least three fragrance ingredients of the high performance fragrance composition have an intensity value greater than 60. As used herein, the term “fragrance intensity,” “perceived intensity,” “perceived fragrance intensity,” “perceived fragrance performance” or “perceived performance” are used interchangeably to refer to the intensity of a fragrance as perceived by a consumer. Such odor characteristics of a fragrance composition are typically assessed under different conditions by trained panelists that are capable of differentiating unambiguously the odor of a given fragrance composition under a first condition, for example during or after dilution of a rinse-off conditioner product containing said fragrance composition, or on a substrate wetted with the product, from that of the same perfumed product, but under a second condition, for example after the product has dried on the substrate. Under such conditions, the difference is deemed to be consumer noticeable, that is, a majority of consumers will perceive the change of odor from said first condition to said second condition.

For the purposes of this invention, fragrance intensity is rated on a scale ranging from 0 to 100 thereby providing a fragrance intensity value or score. In particular aspects, fragrance intensity is determined at a dry stage, e.g., after applying the fragrance to wet hair and subsequently drying the hair. In other aspects, fragrance intensity is determined 24 hours after drying. In still further aspects, fragrance intensity is determined on a substrate, e.g., hair samples, at a dosage of 0.2 wt % in a rinse-off conditioner product. In accordance with such a rating, a fragrance intensity value of 4 would indicate that the fragrance on the substrate being evaluated produces a weak intensity while an intensity value of 70 would indicate that the fragrance on the substrate generates a very strong smell.

Fragrance intensity indices can also be used to rate the fragrance intensity in a fragrance composition or a consumer product containing the same (together referred to as “sample”). The fragrance intensity index is the ratio between (i) the sensory intensity score of a sample and (ii) the sensory intensity score of allyl amyl glycolate (AAG), as the standard. The sensory intensity score of AAG is scaled at a range of 0 to 100 evaluated by a sensory panel when AAG is dissolved in an appropriate solvent (e.g., diethyl phthalate). The sample is also evaluated by the same panel at the same concentration, preferably under the same conditions and in the same sensory evaluation study conducted within the same day. The fragrance intensity index is then calculated as: sensory intensity score of a sample/sensory intensity score of AAG. See, e.g., US2020/0046616 A1 and U.S. Pat. No. 9,162,085 B2.

The fragrance ingredients, standard, accords, fragrance compositions, and rinse-off conditioner products described herein each can be evaluated as described above and can also be evaluated following protocols well-known in the art using different scales, which can be normalized to a scale of 0-100 for calculating the fragrance intensity values.

In accordance with the present invention, the fragrance composition includes at least two fragrance ingredients having, on a scale of 0 to 100, an intensity value in the range of 40 to 60. In certain aspects, the fragrance composition more preferably includes at least two, preferably three, or more preferably four fragrance ingredients having an intensity value in the range of 40 to 60. In other aspects, the fragrance composition includes at least two fragrance ingredients having an intensity value in the range of 40 to 60, wherein each of the fragrance ingredients is present at a dosage greater than 2 wt % of the fragrance composition. In certain aspects, the fragrance composition includes at least two fragrance ingredients having an intensity value in the range of 40 to 60, wherein each of the fragrance ingredients is present at a dosage greater than 2 wt %, preferably 3 wt %, more preferably 4 wt % or most preferably 5 wt % of the fragrance composition. In some aspects, the fragrance composition includes at least two, three, or four fragrance ingredients having an intensity value in the range of 40 to 60, wherein each of said fragrance ingredients is present at a dosage greater than 2 wt %, 3 wt %, 4 wt % or 5 wt % of the fragrance composition. In other aspects, the fragrance composition includes at least two, three, or four fragrance ingredients having an intensity value in the range of 40 to 60, wherein each of said fragrance ingredients is present at a dosage of no greater than 10 wt %, 9 wt %, 8 wt %, 7 wt %, 6 wt % or 5 wt % of the fragrance composition.

The present invention further provides that the fragrance composition includes at least three fragrance ingredients having, on a scale of 0 to 100, an intensity value that is greater than 60. In certain aspects, the fragrance composition more preferably includes at least three, preferably four or more preferably five fragrance ingredients having an intensity value that is greater than 60. In other aspects, the fragrance composition includes at least three fragrance ingredients having an intensity value that is greater than 60, wherein each of the fragrance ingredients is present at a dosage greater than 1 wt % of the fragrance composition. In certain aspects, the fragrance composition includes at least three fragrance ingredients having an intensity value that is greater than 60, wherein each of the fragrance ingredients is present at a dosage greater than 1 wt %, preferably 2 wt %, more preferably 3 wt %, or most preferably 4 wt % of the fragrance composition. In particular aspects, the fragrance composition includes at least three, four or five fragrance ingredients having an intensity value that is greater than 60, wherein each of the fragrance ingredients is present at a dosage greater than 1 wt %, 2 wt %, 3 wt %, or 4 wt % of the fragrance composition. In other aspects, the fragrance composition includes at least three, four or five fragrance ingredients having an intensity value that is greater than 60, wherein each of said fragrance ingredients is present at a dosage of no greater than 10 wt %, 9 wt %, 8 wt %, 7 wt %, 6 wt % or 5 wt % of the fragrance composition.

The intensity values of the fragrance ingredients in Table 1 are presented in Table 2.

TABLE 2

| | | Intensity |
|---|---|---|
| Fragrance Ingredient | CAS NO. | Value |
| DIMETH BENZ CARB | 10094-34-5 | 40 |
| BUTY | | |
| ROSETONE | 90-17-5 | 40 |
| BACDANOL | 28219-61-6 | 43 |
| CYCLAMAL | 103-95-7 | 44 |
| BOURGEONAL | 18127-01-0 | 47 |
| LILIAL | 80-54-6 | 48 |
| ALD C-16 | 77-83-8 | 49 |
| STARFLEUR | 56836-93-2 | 50 |
| ALD C-12 MNA | 110-41-8 | 50 |
| ARCTICAL | 61668-40-4 | 50 |
| DECALACTONE | 706-14-9 | 52 |
| GAMMA | | |
| THYMOL | 89-83-8 | 53 |
| OPALENE | 174155-47-6 | 54 |
| VANITROPE | 94-86-0 | 54 |
| SALICYNALVA | 3508-98-3 | 54 |
| GALBASCONE | 56973-85-4 | 55 |
| BENZ BENZOATE | 120-51-4 | 57 |
| PATCHOULI OIL | 9/3/8014 | 58 |
| CYCLAPROP | 67634-24-6 | 61 |
| KHUSINIL | 75490-39-0 | 62 |
| INDOLE | 120-72-9 | 63 |
| UNDECALACTONE | 104-67-6 | 64 |
| GAMMA | | |
| DAMASCONE ALPHA | 24720-09-0 | 64 |
| ALD C-11 INTRELEVEN | 143-14-6 | 67 |
| RHUBOFIX | 41816-03-9 | 68 |
| PHARAONE | 313973-37-4 | 69 |
| DAMASCONE DELTA | 71048-82-3 | 70 |
| AMYL CINN ALD | 78605-96-6 | 73 |
| ETH-2,4-DECADIENOATE | 3025-30-7 | 76 |
| ISO BUTYL QUINOLINE | 65442-31-1 | 80 |
| ALD C-12 LAURIC | 112-54-9 | 83 |

In some aspects, the high performance fragrance composition includes at least 2 wt % DIMETH BENZ CARB BUTY, at least 2 wt % BACDANOL, at least 1 wt % CYCLAPROP, at least 1 wt % UNDECALACTONE GAMMA and at least 1 wt % AMYL CINN ALD, based upon the total weight of the high performance fragrance composition.

In addition to the high performance fragrance ingredients listed in Tables 1 and 2, the fragrance composition may further include at least 1, 2, 3, 4, 5, 6, 7, 8, 9, 10 or more additional fragrance ingredients. In some aspects, the fragrance composition includes at least 10, at least 15, at least 20, at least 25, at least 30, at least 35, at least 40, at least 45, and/or at least 50 additional fragrance ingredients. The more fragrance raw materials that make up a fragrance composition, the more complex the fragrance composition is considered to be.

Additional fragrance ingredients, if not already included as a high performance fragrance ingredient, may include:
(i) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, cistus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue chamomile oil, Roman chamomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil, spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniperberry oil, wine lees oil, wormwood oil, Wintergreen oil, ylang-ylang oil, hyssop oil, civet absolute, cinnamon leaf oil, cinnamon bark oil, and fractions thereof or ingredients isolated therefrom;
(ii) hydrocarbons such as, for example, 3-carene, a-pinene, β-pinene, a-terpinene, γ-terpinene, p-cymene, bisabolene, camphene, caryophyllene, cedrene, farnesene, limonene, longifolene, myrcene, ocimene, valencene, (E, Z)-1,3,5-undecatriene, styrene, and diphenylmethane;
(iii) aliphatic alcohols such as, for example, hexanol, octanol, 3-octanol, 2,6-dimethyl-heptanol, 2-methyl-2-heptanol, 2-methyl-2-octanol, (E)-2-hexenol, (E)- and (Z)-3-hexenol, 1-octen-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;
(iv) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetra-methyl-6-octen-3-one, aliphatic sulfur-containing compounds, such as for example 3-methylthio-hexanol, 3-methylthiohexyl acetate, 3-mercaptohexanol, 3-mercaptohexyl acetate, 3-mercapto-hexyl butyrate, 3-acetylthiohexyl acetate, 1-menthene-8-thiol, and aliphatic nitriles (e.g., 2-nonenenitrile, 2-tridecenenitrile, 2,12-tridecenenitrile, 3,7-dimethyl-2,6-octadienenitrile, and 3,7-dimethyl-6-octenenitrile);
(v) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octen-3-yl acetate, ethyl butyrate, butyl butyrate, isoamyl butyrate, hexylbutyrate, (E)- and (Z)-3-hexenyl isobutyrate, hexyl crotonate, ethylisovate rate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;
(vi) acyclic terpene alcohols, such as, for example, citronellol, geraniol, nerol, linalool, lavandulol, nerolidol, farnesol, tetrahydrolinalool, tetrahydrogeraniol, 2,6-dimethyl-7-octen-2-ol, 2,6-dimethyloctan-2-ol, 2-methyl-6-methylene-7-octen-2-ol, 2,6-dimethyl-5,7-octadien-2-ol, 2,6-dimethyl-3,5-octadien-2-ol, 3,7-dimethyl-4,6-octadien-3-ol, 3,7-dimethyl-1,5,7-octatrien-3-ol, 2,6-dimethyl-2,5,7-octatrien-1-ol, as well as formates, acetates, propionates, isobutyrates, butyrates, isovalerates, pentanoates, hexanoates, crotonates, tiglinates and 3-methyl-2-butenoates thereof;
(vii) acyclic terpene aldehydes and ketones, such as, for example, geranial, neral, citronellal, 7-hydroxy-3,7-dimethyloctanal, 7-methoxy-3, 7-dimethyloctanal, 2,6,10-trimethyl-9-undecenal, α-sinensal, β-sinensal, geranylacetone, as well as the dimethyl- and diethylacetals of geranial, neral and 7-hydroxy-3, 7-dimethyloctanal;
(viii) cyclic terpene alcohols, such as, for example, menthol, isopulegol, alpha-terpineol, terpinen-4-ol, menthan-8-ol, menthan-1-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;
(ix) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethyl ionone, β-isomethylionone, α-irone, α-damascone, β-damascone, β-damascenone, 8-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2, 4a-methanonaphthalen-8(5H-)-one, nootkatone, dihydronootkatone; acetylated cedarwood oil (cedryl methyl ketone);
(x) cyclic alcohols, such as, for example, 4-tert-butylcyclohexanol, 3,3,5-trimethylcyclo-hexanol, 3-isocamphylcyclohexanol, 2,6,9-trimethyl-Z2,Z5,E9-cyclododecatrien-1-ol, 2-iso-butyl-4-methyltetrahydro-2H-pyran-4-ol;

(xi) cycloaliphatic alcohols, such as, for example, alpha, 3,3-trimethylcyclo-hexylmethanol, 2-methyl-4-(2,2,3-trimethyl-3-cyclopent-1-yl) butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl) pentan-3-ol, 1-(2,2,6-trimethylcyclohexyl) hexan-3-ol;
(xii) cyclic and cycloaliphatic ethers, such as, for example, cineole, cedryl methyl ether, cyclododecyl methyl ether;
(xiii) (ethoxymethoxy)cyclododecane; alpha-cedrene epoxide, 3a,6,6,9a-tetramethyl-dodecahydronaphtho[2,1-b]furan, 3a-ethyl-6,6,9a-trimethyldodecahydronaphtho[2,1-b]furan, 1,5,9-trimethyl-13-oxabicyclo[10.1.0]-trideca-4, 8-diene, rose oxide, 2-(2,4-dimethyl-3-cyclohexen-1-yl)-5-methyl-5-(1-methylpropyl)-1,3-dioxan;
(xiv) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-5-cyclopentadecenone, 3-methylcyclopentadecanone, 4-(1-ethoxyvinyl)-3,3,5,5-tetramethylcyclohexanone, 4-tert-pentylcyclohexanone, 5-cyclohexadecen-1-one, 6,7-dihydro-1,1,2,3,3-pentamethyl-4 (5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4 (2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;
(xv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl) ketone;
(xvi) esters of cyclic alcohols, such as, for example, 2-tert-butylcyclohexyl acetate, 4-tert-butylcyclohexyl acetate, 2-tert-pentyl cyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;
(xvii) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-dioxolane-2-acetate;
(xviii) aromatic and aliphatic alcohols, such as, for example, benzyl alcohol, 1-henylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl) propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl) ethanol;
(xix) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, α,α-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin, 4,4a,5,9b-tetrahydro-2,4-dimethylindeno[1,2-d]-m-dioxin;
(xx) aromatic and aliphatic aldehydes, such as, for example, benzaldehyde; phenylacetaldehyde, 3-phenylpropanal, hydratropaldehyde, 4-methylbenzaldehyde, 4-methylphenyl-acetaldehyde, 3-(4-ethylphenyl)-2,2-dimethylpropanal, 2-methyl-3-(4-isopropylphenyl) propanal, 2-methyl-3-(4-tert-butylphenyl) propanal, 3-(4-tert-butylphenyl) propanal, cinnamaldehyde, α-butylcinnamaldehyde, α-amylcinnamaldehyde, α-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl propanal, 2-methyl-3-(4-methylendioxyphenyl) propanal;
(xxi) aromatic and aliphatic ketones, such as, for example, acetophenone, 4-methylaceto-phenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl) ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methyl-ethyl)-1H-5-indenylJethanone, 5′, 6′, 7′, 8′-tetrahydro-3′, 5′, 5′, 6′, 8′, 8′-hexamethyl-2-acetonaphthone;
(xxii) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3, 6-dimethylbenzoate, ethyl 3-phenylglycidate, ethyl 3-methyl-3-phenylglycidate;
(xxiii) nitrogen-containing aromatic compounds, such as, for example, 2,4,6-trinitro-1, 3-dimethyl-5-tert-butylbenzene, 3,5-dinitro-2, 6-dimethyl-4-tert-butylacetophenone, cinnamonitrile, 5-phenyl-3-methyl-2-pentenonitrile, 5-phenyl-3-methylpentanonitrile, methyl anthranilate, methyl-N-methylanthranilate, Schiffs bases of methyl anthranilate with 7-hydroxy-3, 7-dimethyl-octanal, 2-methyl-3-(4-tert-butylphenyl) propanal or 2,4-dimethyl-3-cyclohexene carbaldehyde, 6-isopropylquinoline, 6-isobutylquinoline, 6-sec-butylquinoline, indole, skatole, 2-methoxy-3-isopropylpyrazine, 2-isobutyl-3-methoxypyrazine;
(xxiv) phenols, phenyl ethers and phenyl esters, such as, for example, estragole, anethole, eugenol, eugenyl methyl ether, isoeugenol, isoeugenol methyl ether, thymol, carvacrol, diphenyl ether, β-naphthyl methyl ether, β-naphthyl ethyl ether, β-naphthyl isobutyl ether, 1,4-dimethoxybenzene, eugenyl acetate, 2-methoxy-4-methylphenol, 2-ethoxy-5-(1-propenyl) phenol, p-cresyl phenylacetate;
(xxv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;
(xxvi) lactones, such as, for example, 1,4-octanolide, 3-methyl-1, 4-octanolide, 1,4-nonanolide, 1,4-decanolide, 8-decen-1, 4-olide, 1,4-undecanolide, 1,4-dodecanolide, 1,5-decanolide, 1,5-dodecanolide, 1,15-pentadecanolide, cis- and trans-11-pentadecen-1, 15-olide, cis- and trans-12-pentadecen-1, 15-olide, 1,16-hexadecanolide, 9-hexadecen-1, 16-olide, 10-oxa-1, 16-hexadecanolide, 11-oxa-1, 16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and
(xxvii) malodor counteracting agents including an α,β-unsaturated carbonyl compounds including but not limited to those disclosed in U.S. Pat. No. 6,610,648 and EP2,524,704, amyl cinnamaldehyde, benzophenone, benzyl benzoate, benzyl isoeugenol, benzyl phenyl acetate, benzyl salicylate, butyl cinnamate, cinnamyl butyrate, cinnamyl isovalerate, cinnamyl propionate, decyl acetate, ethyl myristate, isobutyl cinnamate, isoamyl salicylate, phenethyl benzoate, phenethyl phenyl acetate, triethyl citrate, tripropylene glycol n-butyl ether, isomers of bicyclo[2.2.1]hept-5-ene-2-carboxylic acid, ethyl ester, nano silver, zinc undecenylate, β-naphthyl methyl ether, β-naphthyl ketone, benzyl acetone. They may include mixture of hexahydro-4,7-methanoinden-5-yl propionate and hexahydro-4,7-methanoinden-6-yl propionate; 4-(2,6,6-trimethyl-2-cyclohexen-1-yl)-3-methyl-3-buten-2-one; 3,7-dimethyl-2,6-nonadien-1-nitrile; dodecahydro-3a,6,6,9a-tetramethylnaphtho (2,1-b) furan; ethylene glycol cyclic ester of n-dodecanedioic acid; 1-cyclohexadecen-6-one; 1-cycloheptadecen-10-one; and corn mint oil. They may also include 1-cyclohexylethan-1-yl butyrate; 1-cyclohexylethan-1-yl acetate; 1-cyclohexylethan-1-ol; 1-(4′methylethyl)cyclohexylethan-1-yl propionate; and 2′-hydroxy-1′-ethyl(2-phenoxy) acetate each of which compound is marketed under the trademark VEILEX by International Flavors & Fragrances Inc. More suitable malodor counteracting agents are polymers containing an α-keto, benzaldehyde, or α, β-unsaturated carbonyl moiety, such as those described in US2012/0294821, US2013/0101544 and US2013/0101545.

Additional fragrance ingredients are described in, e.g., Arctander (2000) Perfume Flavors and Chemicals. Vols. I and II, Montclair, NJ; The Merck Index, 8^{th} Edition (1968) Merck & Co., Inc. Rahway, NJ; Allured's Flavor and Fragrance Materials (2015) Allured Publishing Corp.

The fragrance composition of this invention can be used as a neat fragrance and/or be encapsulated in a delivery system. A wide variety of encapsulating materials exist which allow for delivery of fragrances at various times in the cleaning or conditioning process. Suitable encapsulating materials include starches, oligosaccharides, cyclodextrins, polyethylenes, polyamides, polystyrenes, polyisoprenes, polycarbonates, polyesters, polyacrylates, vinyl polymers polyurethanes, amorphous silica, precipitated silica, fumed silica, aluminosilicates, such as zeolites and alumina, and mixtures thereof.

The fragrance composition of this invention finds particular use in a hair rinse-off conditioner. Hair rinse-off conditioner products including the high performance fragrance composition of the invention will also include a product base, which fulfills one or more specific actions, such as cleaning, softening, and caring or the like. Components of the hair product base may include any suitable additive that produces an intended effect under intended use conditions of the hair care product. For example, hair product base ingredients may be selected from hair conditioning agents, skin care agents, sunscreen agents, and skin conditioning agents. One of ordinary skill in the art will appreciate that the hair product base ingredients will be selected based upon the desired intended use of the hair product. For example, a hair conditioning product may include one or more suitable surfactants, silicones, chelants, dyes, pigments, or formaldehyde scavengers to provide the desired benefit to a consumer.

Surfactants utilized may be of the anionic, nonionic, zwitterionic, ampholytic or cationic type or may comprise compatible mixtures of these types. The product may contain up to from 0.01% to about 30%, alternatively from about 0.01% to about 20%, more alternatively from about 0.1% to about 10%, by weight of the product, of a surfactant.

Suitable silicones include Si—O moieties and may be selected from (a) non-functionalized siloxane polymers, (b) functionalized siloxane polymers, and combinations thereof. The molecular weight of the organosilicone is usually indicated by the reference to the viscosity of the material. In one aspect, the organosilicones may include a viscosity of from about 10 to about 2,000,000 centistokes at 25° C. In another aspect, suitable organosilicones may have a viscosity of from about 10 to about 800,000 centistokes at 25° C.

Suitable organosilicones may be linear, branched or cross-linked. In some examples, the organosilicone may be a cyclic silicone. The cyclic silicone may be a cyclomethicone of the formula [(CH₃)₂SiO]ₙ where n is an integer that may range from about 3 to about 7, or from about 5 to about 6.

In some examples, the organosilicone may include a functionalized siloxane polymer. Functionalized siloxane polymers may include one or more functional moieties selected from the group of amino, amido, alkoxy, hydroxy, polyether, carboxy, hydride, mercapto, sulfate phosphate, and/or quaternary ammonium moieties. These moieties may be attached directly to the siloxane backbone through a bivalent alkylene radical, (i.e., “pendant”) or may be part of the backbone. Suitable functionalized siloxane polymers include materials selected from the group consisting of aminosilicones, amidosilicones, silicone polyethers, silicone-urethane polymers, quaternary ABn silicones, amino ABn silicones, and combinations thereof.

In some examples, the functionalized siloxane polymer may include a silicone polyether, also referred to as “dimethicone copolyol.” In general, silicone polyethers include a polydimethylsiloxane backbone with one or more polyoxyalkylene chains. The polyoxyalkylene moieties may be incorporated in the polymer as pendent chains or as terminal blocks. In some examples, the functionalized siloxane polymer may include an aminosilicone.

The product may contain less than about 5%, or from about 0.01% to about 3%, by weight of the product, of a chelant such as citrates; nitrogen-containing, P-free aminocarboxylates such as EDDS, EDTA and DTPA; aminophosphonates such as diethylene triamine pentamethylenephosphonic acid and, ethylenediamine tetramethylenephosphonic acid; nitrogen-free phosphonates e.g., HEDP; and nitrogen or oxygen containing, P-free carboxylate-free chelants.

The hair product can contain any fragrance composition described herein at a level of 0.01 wt % to 20 wt % (e.g, 0.02 wt % to 10 wt %, 0.05 wt % to 5 wt %, 0.05 wt % to 2 wt %, 0.1 wt % to 0.5 wt %), wherein the lower limit may be 0.01 wt %, 0.02 wt %, 0.05 wt %, 0.1 wt %, 0.2 wt %, or 0.5 wt %, and the upper limit may be 20 wt %, 15 wt %, 10 wt %, 8 wt %, 6 wt %, 5 wt %, 3 wt %, 2 wt %, 1 wt %, 0.8 wt %, or 0.5 wt %. In certain aspects, the hair rinse-off conditioner includes between 0.5 wt % and 1.2 wt % of the high performance fragrance composition.

As will be appreciated by the skilled artisan, fragrance ingredients meeting the criteria of formula (I) and intensity values set forth herein may vary depending on the product and/or product base components in which the fragrance ingredients are to be included in.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%” and encompasses, e.g., 50±5%.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8,” “3 to 10,” “2 to 7,” “1.5 to 6,” “3.4 to 7.8,” “1 to 2 and 7-10,” “2 to 4 and 6 to 9,” “1 to 3.6 and 7.2 to 8.9,” “1-5 and 10,” “2 and 8 to 10,” “1.5-4 and 8,” and the like.

The terms “g,” “mg,” and “μg” refer to “gram,” “milligram,” and “microgram,” respectively. The terms “L” and “mL” refer to “liter” and “milliliter,” respectively.

The invention is described in greater detail by the following non-limiting examples. Without further elaboration, it is believed that one skilled in the art can, based on the description herein, utilize the present invention to its fullest extent. All publications cited herein are incorporated by reference in their entirety.

### Example 1: Analytical Measurement of Fragrance Recovery

Preparation of Internal Standard Solution. Ethyl tetradecanoate (0.1 g) was placed in a 100 mL flask and ethanol was added to prepare the ethyl tetradecanoate standard.

Preparation of Standard Solutions. A stock solution was prepared by weighing 10*m of neat perfume in a 100 mL flask (“m” is the theoretical weight of deposited perfume) and q.s. with ethanol, “m” was equal to the weight of product applied on hair (mg)×dosage of fragrance in product (%).

In addition, three standard solutions representing 10%, 5%, and 2% of recovery were prepared by respectively adding 1000 μL, 500 μL and 200 μL of stock solution in 20 mL flasks, adding 200 μL of the internal standard and q.s. with ethanol.

Extraction of Perfume on Hair. A hair swatch was placed in a 30 mL container. Ethanol (20 mL) and 200 μL of the internal standard (ethyl tetradecanoate) were added to the hair swatch. Each container was closed and placed in an ultrasound bath for 30 minutes and subsequently transferred to an agitation plate for 30 minutes. After agitation, a 2 mL gas chromatography vial was filled with extract. The standard solutions along with three individual samples for each hair sample were analyzed for fragrance deposition by injecting the sample into a GC-MS using a SIM acquisition method. Standard solutions were injected into the GC-MS using the same acquisition method.

Quantification. Fragrance recovery was determined by quantification of all perfumery ingredients using a calibration curve obtained from the standard solutions.

### Example 2: Fragrance Intensity Determinations

Ingredients were dosed at 0.2% in shampoo and at 0.2% in conditioner. Synthetic hair swatches were used for this analysis.

Application Protocol on Hair Swatches. Hair swatches were immersed in cold water for 2 minutes. Shampoo (2 g) was applied to the swatch and the hair was turned 10 turns in a circular motion in one direction then 10 in the other direction. The hair swatch was smoothed 5 times from top to bottom and subsequently allowed to rest for 5 minutes. The swatch was rinsed for 30 seconds horizontally and 2 g of conditioner was subsequently applied. Again, the swatch was turned 10 turns in a circular motion in one direction then 10 in the other direction, smoothed 5 times from top to bottom, allowed to rest for 5 minutes and rinsed for 30 seconds horizontally. The hair swatch was wrung out to remove excess water and hung up to dry overnight.

Evaluation on Hair Swatches. After drying for 24 hours, a trained panel of at least 15 people evaluated the intensity of the fragrance on the dry hair swatches as compared to Allyl Amyl Glycolate (AAG) as a reference, which was equal to 50 on the 0 to 100 intensity scale.

### Example 3: Protocol for Assessing Fragrance Performance

Application Protocol on Hair Swatches. A hair conditioner base was dosed with between 0.4 wt % to 1.4 wt % a fragrance composition of the invention. A natural hair swatch (8 g) was wet under a stream of water (25° C., flow rate of 2 L/min) for 1 minute. The hair swatch was gently squeezed using two fingers to remove excess water. The hair swatch was placed in a Petri dish residing on a balance and 2 g of conditioner were applied to the hair swatch. The hair was lathered between the palms clockwise for 10 turns followed by 10 counterclockwise circular motions. The hair of the swatch was detangled and placed on a Petri dish for 15 seconds to allow the fragrance in the condition to be absorbed by the hair. Holding the swatch in both hands, horizontally, the swatch was rinsed under a stream of water (25° C., flow rate of 2 L/min) while slowly moving the swatch side to side for 20 seconds. Using two fingers, the hair swatch was gently squeezed to removed excess water.

Evaluation on Hair Swatches. After drying for 24 hours, an untrained panel of 15 people evaluated the intensity of the fragrance on the dry hair swatches.

### Example 4: Performance of a Fragrance Formulation Including High Performance Fragrance Ingredients

To demonstrate the effect of the fragrance composition of the invention in improving performance on dry hair, several different fragrance compositions and comparative compositions were prepared (Table 3). Accord AA (a reference accord) contained two ingredients with an intensity of >60 at a dosage >1% (i.e., CYCLAPROP and GAMMA DECALACTONE) and one ingredient with an intensity >40 at a dosage at 2% (i.e., CYCLAMAL), with additional base ingredients that were retained at a constant amount across all accords (i.e., HEXENOL CIS-3, VERDOX, PHENOXY ETHYL ISO BUTYRATE, ALLYL CYCLOHEXYL PROP, IONONE BETA EXTRA, HELIONAL, DAMASCENONE, KHARISMAL). Accords AC and AC′ contained three or four ingredients with an intensity >60 at a dosage >1% (i.e., CYCLAPROP, GAMMA DECALACTONE, DAMASCENONE DELTA, AMYL CINN ALD COEUR) and two or three ingredients with an intensity >40 at a dosage >2% (i.e., CYCLAMAL, XYPHENYLON and LILIAL). Accords AE and AD each lacked the number and/or dosage of fragrances required by the method of the present invention.

TABLE 3

| | | Amount (grams) | | | | |
|---|---|---|---|---|---|---|
| Fragrance | Intensity | AA | AC | AC′ | AD | AE |
| HEXENOL CIS-3 | 4 | 2 | 2 | 2 | 2 | 2 |
| VERDOX | 11 | 60 | 60 | 60 | 60 | 60 |
| PHENOXY ETHYL | 14 | 20 | 20 | 20 | 20 | 20 |
| ISO BUTYRATE | | | | | | |
| ALLYL CYCLOHEXYL | 20 | 9 | 9 | 9 | 9 | 9 |
| PROP | | | | | | |
| IONONE BETA | 26 | 2.5 | 2.5 | 2.5 | 2.5 | 2.5 |
| EXTRA | | | | | | |
| HELIONAL | 28 | 7.5 | 7.5 | 7.5 | 7.5 | 7.5 |
| DAMASCENONE | 35 | 0.9 | 0.9 | 0.9 | 0.9 | 0.9 |
| KHARISMAL | 38 | 2.5 | 2.5 | 2.5 | 2.5 | 2.5 |
| METH DH | 32 | 120 | 96.1 | 67.6 | 106 | 110.1 |
| JASMONATE | | | | | | |
| CYCLAMAL | 44 | 29 | 29 | 29 | 29 | 29 |
| OXYPHENYLON | 54 | 6 | 20 | 20 | 20 | 6 |
| LILIAL | 48 | 1 | 1 | 20 | 1 | 1 |
| CYCLAPROP | 61 | 12 | 12 | 12 | 12 | 12 |
| DECALACTONE, | 66 | 14 | 14 | 14 | 14 | 14 |
| GAMMA | | | | | | |
| DAMASCONE DELTA | 67 | 2.5 | 2.5 | 12 | 2.5 | 2.5 |
| AMYL CINN ALD | 77 | 0.1 | 10 | 10 | 0.1 | 10 |
| COEUR | | | | | | |
| Water | — | 711 | 711 | 711 | 711 | 711 |
| Total | — | 1000 | 1000 | 1000 | 1000 | 1000 |
| Intensity Perceived | | 2.3 | 3.3 | 5.6 | 2 | 2.1 |
| at 24 h | | | | | | |

The AC, AC′, AD and AE accords were compared to the reference accord AA. Each accord was applied at 1% in the hair conditioner base presented in Table 4. The intensity of each accord was assessed by a naive panel (N=12) on swatches after 24 hours.

The results of this analysis indicated that the perceived intensity of each of Accords AC and AC′ was significantly higher than that of the reference AA accord. By comparison, the perceived intensities of Accords AD and AE were similar to the reference AA accord.

### Example 5: Hair Conditioner Formulations

High performance fragrance compositions are formulated into a rinse-off hair conditioner. Exemplary rinse-off hair conditioner base formulations are presented in Tables 4 and 5.

TABLE 4

| Ingredient | % w/w |
|---|---|
| Water (Aqua) | QS 100 |
| Disodium EDTA (BASF) | 0.10 |
| Cetyl alcohol (BASF) | 1.50 |
| Stearyl alcohol (BASE) | 2.00 |
| Behentrimonium methosulfate | 6.00 |
| (Croda) | |
| Bis hydroxy/methoxy | 3.00 |
| amodimethicone (Dow) | |
| Methychloroisothiazolinone (and) | 0.10 |
| Methylisothiazolinone (Dow) | |
| Panthenol (DSM) | 0.30 |
| Panthenyl Ethyl Ether (DSM) | 1.00 |
| Citric acid (30% solution in | 0.15 |
| water) | |

pH 4.3 ± 0.2; Viscosity 180000-270000 cPs.

TABLE 5

| | | % Ingredient in |
|---|---|---|
| Ingredient | Range | formulation |
| Water (Aqua) | QS 100 | QS 100 |
| Cetearyl alcohol | 1%-8% | 3.00 |
| Stearamidopropyl Dimethylamine | 0%-4% | 0.80 |
| (TAS) | | |
| Behentrimonium Chloride (BTAC) | 0%-4% | 0.60 |
| Cetrimonium Chloride (CTAC) | 0%-4% | — |

What is claimed is:

1. A high performance fragrance composition comprising at least five fragrance ingredients each having an analytical deposition value and retention value, wherein the combined analytical deposition value and retention value of each fragrance falls with a range set forth by formula (I):
$$60 \% \leq R + 5 D \leq 150 \% , \qquad ( I )$$
wherein “R” is the experimentally determined percent retention of the fragrance on a dry hair sample after 10 hours and “D” is an experimentally determined percent deposition of the fragrance on a wet hair sample, wherein the at least five fragrances comprise:
(i) at least two fragrance ingredients having an intensity value in the range of 40 to 60, wherein each of the fragrance ingredients is present at a dosage greater than 2 wt % of the high performance fragrance composition, and
(ii) at least three fragrance ingredients having an intensity value greater than 60, wherein each of the fragrance ingredients is present at a dosage greater than 1 wt % of the high performance fragrance composition,
wherein the intensity value of (i) and (ii) is determined on a scale of 0 to 100.

2. The high performance fragrance composition of claim 1, wherein the composition comprises (i) at least two to four fragrance ingredients having an intensity value in the range of 40 to 60, wherein each of the fragrance ingredients is present at a dosage greater than 2 wt % of the fragrance composition.

3. The high performance fragrance composition of claim 1, wherein the composition comprises (i) at least two to four fragrance ingredients having an intensity value in the range of 40 to 60, wherein each of said fragrance ingredients is present at a dosage greater than 5 wt % of the fragrance composition.

4. The high performance fragrance composition of claim 1, wherein the composition comprises (ii) at least three to five fragrance ingredients having an intensity value greater than 60, wherein each of the fragrance ingredients is present at a dosage greater than 1 wt % of the fragrance composition.

5. The high performance fragrance composition of claim 1, wherein the composition comprises (ii) at least two to four fragrance ingredients having an intensity value greater than 60, wherein each of the fragrance ingredients is present at a dosage greater than 4 wt % of the fragrance composition.

6. A hair rinse-off conditioner product comprising a product base and a high performance fragrance composition of claim 1.
